(19) United States
(12) Patent Application Publication
(10) Pub. No.: US 2012/0190986 A1
(43) Pub. Date: Jul. 26, 2012

(54) ULTRASOUND PROBE AND ULTRASOUND DIAGNOSTIC APPARATUS

(75) Inventor: Tomoo Sato, Kanagawa (JP)

(73) Assignee: FUJIFILM CORPORATION, Tokyo (JP)

(21) Appl. No.: 13/337,925

(22) Filed: Dec. 27, 2011

(30) Foreign Application Priority Data
Jan. 25, 2011 (JP) 2011-012773

(51) Int. Cl. A61B 8/00

(52) U.S. Cl. 600/459

(57) ABSTRACT

An ultrasound probe comprises a transducer array composed of a plurality of arrayed subdice elements, a plurality of signal lines for connecting the transducer array to an apparatus body that controls transmission and reception of ultrasonic waves, and a channel forming/connecting section that selects a connection between the plurality of subdice elements to form a plurality of channels each composed of a selected number of subdice elements which is changed by switching connections between a plurality of subdice elements, and assigns the plurality of channels to any of the plurality of signal lines to select, from among the plurality of signal lines, effective signal lines connected to the plurality of channels to transmit driving signals supplied to the transducer array and reception signals outputted from the transducer array to the apparatus body.

### BACKGROUND OF THE INVENTION

The present invention relates to an ultrasound probe and an ultrasound diagnostic apparatus and particularly to an ultrasound probe comprising a plurality of channels connected respectively to a plurality of subdice elements and an ultrasound diagnostic apparatus using the same.

Conventionally, ultrasound diagnostic apparatus using ultrasound images are employed in the medical field. Generally, an ultrasound diagnostic apparatus of this kind comprises an ultrasound probe having a built-in transducer array and an apparatus body connected to the ultrasound probe. The apparatus body is connected to an ultrasound probe corresponding to the processing capabilities of an ultrasound transmission/reception circuit built in the apparatus body. The ultrasonic probe transmits ultrasonic waves toward a subject according to driving signals supplied from the ultrasound transmission/reception circuit and receives ultrasonic echoes emitted from the subject, whereupon the reception signals thereof are received by the ultrasound transmission/reception circuit of the apparatus body and electrically processed by the apparatus body to produce an ultrasound image.

In recent years, there have been developed ultrasound diagnostic apparatus of portable type that may be transported to a bed side or to a site where emergency medical care is needed. Of such ultrasound diagnostic apparatus are required reduction in size to pursue ease of operation and convenience, which necessitates reduction of scale of the transmission/reception circuits, necessarily resulting in a reduced image quality. Therefore, there is a demand for ultrasound diagnostic apparatus reduced in size and capable of preventing reduction of image quality.

JP 2006-519684 A, for example, describes an ultrasound diagnostic system wherein a portable ultrasound unit is mounted on a docking cart to perform data processing. A reception signal produced by the portable ultrasonic unit is supplied to the docking cart and processed with a high data processing capability, whereupon an ultrasound image is displayed with a high resolution on the monitor provided on the docking cart.

### SUMMARY OF THE INVENTION

The system described in JP 2006-519684 A, with the portable ultrasound unit mounted on the docking cart, is capable of processing the reception signal with a higher processing capability than the processing capability possessed by the portable ultrasound unit. However, because the scale of the ultrasound transmission/reception circuit differs according to a class of the mounted portable ultrasound unit, there is the need to provide a plurality of ultrasound probes respectively corresponding to the classes of ultrasound units used.

An object of the present invention is to provide an ultrasound probe that resolves such problems of the past and capable of being used with diagnostic apparatus bodies equipped with ultrasound transmission/reception circuits of different sizes.

Another object of the invention is to provide an ultrasound diagnostic apparatus using such ultrasound probe.

An ultrasound probe according to the present invention comprises:

a transducer array composed of a plurality of arrayed subdice elements;

a plurality of signal lines for connecting the transducer array to an apparatus body that controls transmission and reception of ultrasonic waves; and

a channel forming/connecting section that selects a connection between the plurality of subdice elements to form a plurality of channels each composed of a selected number of subdice elements which is changed by switching connections between a plurality of subdice elements, and assigns the plurality of channels to any of the plurality of signal lines to select, from among the plurality of signal lines, effective signal lines connected to the plurality of channels to transmit driving signals supplied to the transducer array and reception signals outputted from the transducer array to the apparatus body.

An ultrasound diagnostic apparatus according to the present invention comprises:

the ultrasound probe described above and

at least one apparatus body including a plurality of transmission and reception circuits respectively connected to the effective signal lines,

wherein switching of connections between the plurality of subdice elements by the channel forming/connecting section is performed according to a total number of transmission and reception circuits of the at least one apparatus body.

### BRIEF DESCRIPTION OF THE DRAWINGS

FIG. 1 is a block diagram illustrating a configuration of an ultrasound diagnostic apparatus according to Embodiment 1 of the invention.

FIG. 2 is a cross section schematically illustrating a configuration of a transducer array provided in an ultrasound probe used in Embodiment 1.

FIGS. 3A and 3B are views illustrating connections of subdice elements where two subdice elements constitute one channel and those where three subdice elements constitute one channel, respectively, in Embodiment 1.

FIGS. 4A and 4B are views illustrating circuit configurations of a channel forming section and a channel connecting section where two subdice elements constitute one channel and those where three subdice elements constitute one channel, respectively, in Embodiment 1.

FIGS. 5A and 5B are views illustrating circuit configurations of the channel forming section and the channel connecting section as switched where two subdice elements constitute one channel and those where three subdice elements constitute one channel, respectively, in Embodiment 1.

FIG. 6 is a view illustrating circuit configurations of the channel forming section and the channel connecting section as switched in a variation of Embodiment 1.

FIG. 7 is a block diagram illustrating a configuration of a part of an ultrasound diagnostic apparatus according to Embodiment 2.

FIGS. 8A and 8B are views illustrating a circuit configuration of a channel forming/connecting section where two subdice elements constitute one channel and that where three subdice elements constitute one channel, respectively, in Embodiment 2.

FIG. 9 is a block diagram illustrating a configuration of an ultrasound diagnostic apparatus according to Embodiment 3.

FIG. 10 is a block diagram illustrating an ultrasound diagnostic apparatus according to a variation of Embodiment 3.

FIG. 11 is a block diagram illustrating a configuration of an ultrasound diagnostic apparatus according to Embodiment 4.

### DETAILED DESCRIPTION OF THE INVENTION

The embodiments of the present invention will be described below based on the appended drawings.

### Embodiment 1

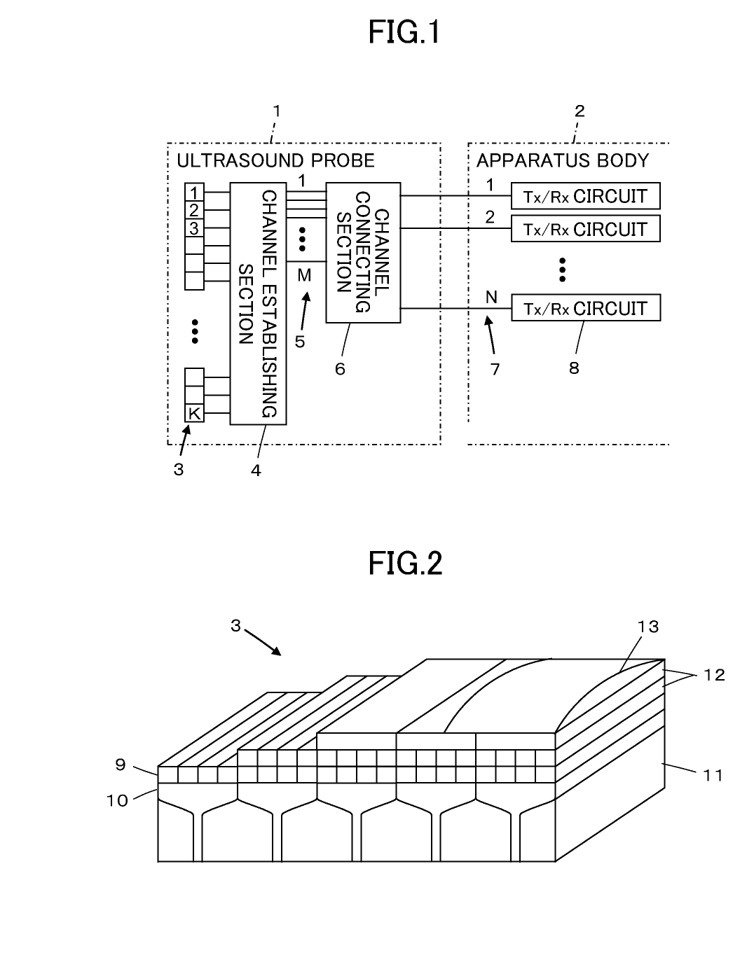

FIG. 1 illustrates a configuration of an ultrasound diagnostic apparatus according to Embodiment 1 of the invention. The ultrasound diagnostic apparatus comprises an ultrasound probe 1 for transmission and reception of ultrasonic waves and a diagnostic apparatus body 2 connected to the ultrasound probe 1.

The ultrasound probe 1 comprises a transducer array 3 including a K number of arrayed subdice elements which forms a plurality of transducers. Each transducer is composed of a given number of subdice elements connected to each other and forms one channel. The transducer array 3 is connected to a channel forming section 4 for forming a plurality of channels each composed of a plurality of subdice elements by selectively switching the connections between the K number of the subdice elements. The channel forming section 4 is connected via an M number of input/output lines 5 to the channel connecting section 6, which in turn is connected to a connection cable comprising an N number of signal lines 7 for transmitting driving signals and reception signals to and from the channel connecting section 6. The channel connecting section 6 is composed of a multiplexer (MUX) to switch connections between the input/output lines 5 and the signal lines 7.

The apparatus body 2 comprises an N number of Tx/Rx (transmission and reception) circuits 8, which are connected to the signal lines 7 of the connection cable. The Tx/Rx circuits 8 supply transmission signals to the respective channels of the ultrasound probe 1 via the signal lines 7 and receive reception signals produced in the respective channels of the ultrasound probe 1. The apparatus body 2 produces image data representing an ultrasound image based on the reception signals received by the Tx/Rx circuits 8.

The number K of the subdice elements of the ultrasound probe 1 is set to be greater than the number M of the input/output lines 5 connecting the channel connecting section 6 and the channel forming section 4, and the number M of the input/output lines 5 is set to be greater than the number N of the signal lines 7 or the Tx/Rx circuits 8.

FIG. 2 illustrates a structure of the transducer array 3 incorporated in the ultrasound probe 1. The transducers constituting the transducer array 3 comprises piezoelectric oscillators 9 in the form of strips constituting a plurality of subdice elements. Each channel of piezoelectric oscillators 9 are provided with an electrode 10 for applying the same voltage. On the rear side of the electrodes are provided damper members 11 for suppressing surplus vibration caused by ultrasonic waves in order to shorten the pulse width of the ultrasonic waves. On the front side of the piezoelectric oscillators 9 are provided acoustic lenses 13 through the intermediary of acoustic matching layers 12.

The piezoelectric oscillators 9 are made of, for example, PZT (Pb (lead) zirconate titanate) or PVDF (polyvinylidene difluoride). Each channel composed of these piezoelectric oscillators 9 is supplied from the electrode 10 with a pulsing voltage or a continuous-wave voltage to cause the piezoelectric oscillators 9 to vibrate in the thickness direction. Because the piezoelectric oscillators 9 in the form of strips constitute the transducers, unnecessary vibrations occurring in other directions than the thickness direction are suppressed. Such vibration causes the individual channels to produce pulsed or continuous-wave ultrasonic waves, which are combined to form an ultrasonic beam. Upon reception of propagating ultrasonic waves, the piezoelectric oscillators 9 constituting the channels contract to produce electric signals. The electric signals are outputted as reception signals of ultrasonic waves.

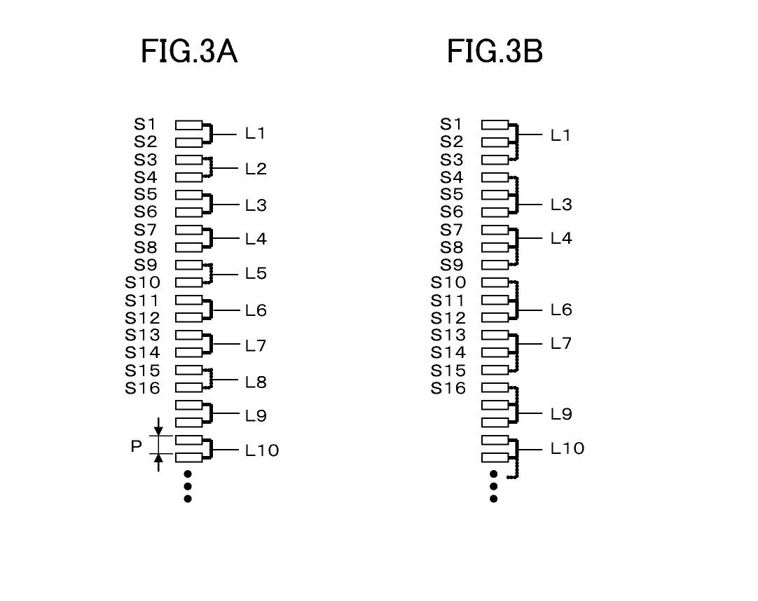

FIGS. 3A and 3B illustrate examples of transducer array 3. The transducer array 3 comprises numerous subdice elements, for example, 384 subdice elements arrayed at equal intervals P of 100 μm and is used at a frequency of about 7.5 MHz. Where each channel is composed of two connected subdice elements, subdice elements S1 and S2, S3 and S4, S5 and S6 . . . are connected to each other, respectively, as illustrated in FIG. 3A, and where each channel is composed of three connected subdice elements, subdice elements S1 to S3, S4 to S6, S7 and S9 . . . are connected to each other, respectively, as illustrated in FIG. 3B.

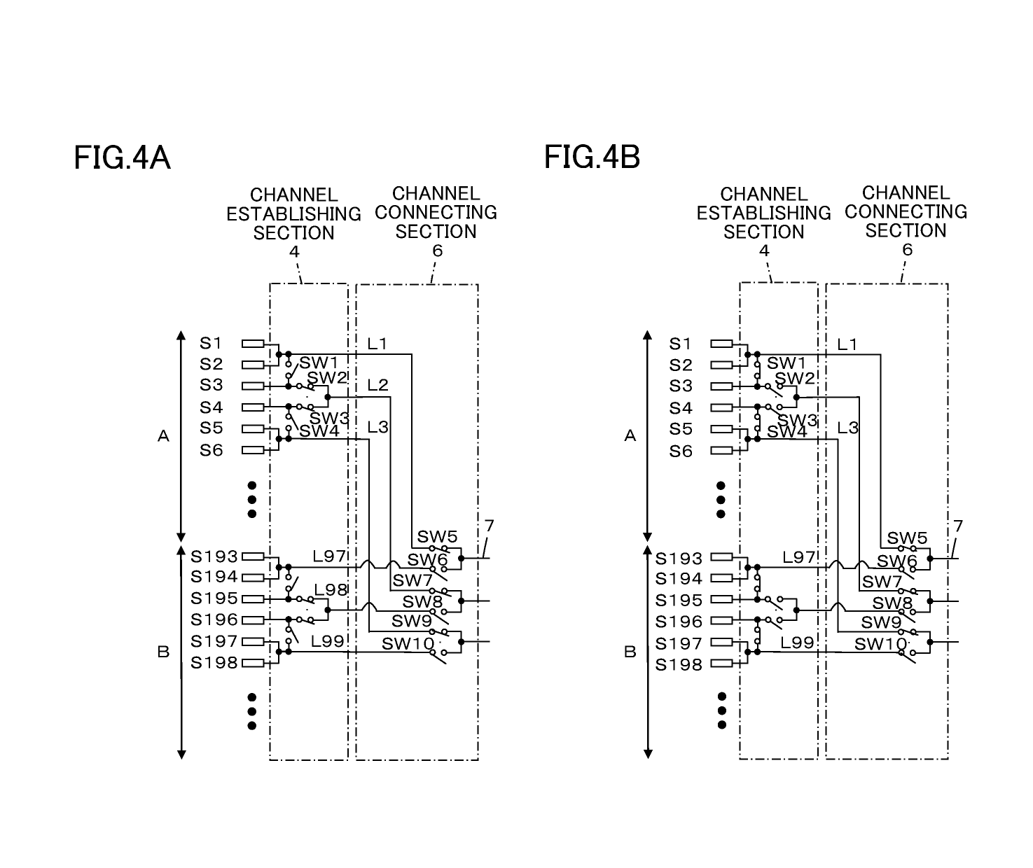

The connections of the subdice elements are so switched by the channel forming section 4 that the number of subdice elements constituting each channel is the same among the channels. Where the connections are so switched that the number of subdice elements constituting each channel is 2 or 3, the channel forming section 4 may be configured by a switch SW1 for switching the connections between the subdice elements S1 and S2 and the subdice element S3, switches SW2 and SW3 for switching the connection between the subdice elements S3 and S4, a switch SW4 for switching the connections between the subdice element S4 and subdice elements S5 and S6, and a plurality of other switches for likewise switching the connections between the subdice elements constituting each of the channels as illustrated in FIGS. 4A and 4B. Thus, the channel forming section 4 may be so configured as to connect the subdice elements S1 and S2, S5 and S6, S7 and S8, S11 and S12, S13 and S14, . . . at all times while switching the connections of the subdice elements S3, S4, S9, S10, . . . .

As illustrated in FIG. 4A, where each channel is composed of two subdice elements, the channel forming section 4 opens the switches SW1, SW4, . . . and closes the switches SW2, SW3, . . . . Thus, 192 channels each composed of two subdice elements are connected to 192 input/output lines L1 to L192 extending from the channel connecting section 6, respectively. On the other hand, where each channel is composed of three subdice elements, the channel forming section 4 closes the switches SW1, SW4, . and opens the switches SW2, SW3, . . . as illustrated in FIG. 4B. Thus, 128 channels each composed of three subdice elements are connected to 128 input/output lines L1, L3, L4, L6, . . . out of 192 of them, respectively. Out of the 192 input/output lines, 64 input/output lines L2, L5, L8, . . . separated from any channels are not connected to the Tx/Rx circuits of the apparatus body 2.

Out of a plurality of channels formed by the channel forming section 4, a given number of channels are sequentially connected by the channel connecting section 6 to a plurality of signal lines 7 extending from the apparatus body 2. Thus, the channel connecting section 6 exclusively assigns two or more channels out of a plurality of channels formed by the channel forming section 4 to the same signal line 7. For example, the channel connecting section 6 used may comprise the switches SW5, SW7, . . . for switching the connections between a plurality of signal lines 7 and the subdice elements S1 to S192 and the switches SW6, SW8, . . . for switching the connections between a plurality of signal lines 7 and the subdice elements S193 to S384 as illustrated in FIGS. 4A and 4B and perform 2:1 switching. Such channel connecting section 6 for 2:1 switching connects an A group of channels composed of a number of channels obtained by equally dividing the number of a plurality of channels formed by the channel forming section 4 to the signal lines 7 and sequentially moves the position of the channels connected to the signal lines 7 by a given number of elements as the ultrasonic waves are transmitted and received. The channel connecting section 6 repeats the process of moving the position of the channels connected to the signal lines 7 until a B group of channels are connected to the signal lines 7. Specifically, when 192 channels are formed by the channel forming section 4, the A group composed of 96 channels to the B group composed of 96 channels are sequentially connected to the signal lines 7, and when 128 channels are formed by the channel forming section 4, the A group composed of 64 channels to the B group composed of 64 channels are sequentially connected to the signal lines 7.

Next, the operation of an ultrasound diagnostic apparatus is described.

First, in FIG. 1, the ultrasound probe 1 where the number K of the subdice elements is 384 and the number M of the input/output lines 5 is 192 is connected to the apparatus body 2 where the number N of the Tx/Rx circuits 8 is 96 via the connection cable containing 96 signal lines 7. The ultrasound probe 1 comprises therein the channel connecting section 6 that performs 2:1 switching.

As illustrated in FIG. 4A, in the ultrasound probe 1 connected to the apparatus body 2, the switches SW1, SW4, . . . are opened and the switches SW2, SW3, . . . are closed in the channel forming section 4 to configure the transducer array 3 composed of 192 channels each connected to two subdice elements. The switches SW5, SW7, . . . are closed and the switches SW6, SW8, . . . are opened in the channel connecting section 6 of the ultrasound probe 1 to connect 96 channels of the A group out of the 192 channels formed by the channel forming section 4 to 96 signal lines.

Because the ultrasound probe 1 thus contains the channel connecting section 6, the number (96) of the signal lines 7 in the connection cable for connecting the ultrasound probe 1 and the apparatus body 2 can be reduced as compared with the number (192) of the input/output lines 5 connected to the channels, and accordingly the ease of operation of the ultrasound probe 1 can be increased.

Upon connection of the ultrasound probe 1 and the apparatus body 2 to each other, 96 Tx/Rx circuits 8 in the apparatus body 2 supplies the driving signals to the channel connecting section 6 of the ultrasound probe 1 via 96 signal lines 7 (effective signal lines). The channel connecting section 6 supplies the channel forming section 4 with the driving signals via the input/output lines L1, L2, . . . , L96 connected to the switches SW5, SW7, . . . . The driving signals supplied to the channel forming section 4 are respectively supplied to the A group composed of 96 channels configured by binding the subdice elements S1 and S2, S3 and S4, . . . , S191 and S192 to each other, respectively. As a result, ultrasonic waves are transmitted from the A group of channels toward a subject (not shown). The ultrasonic echoes reflected by the subject are received by the A group of channels, and the reception signals thereof are inputted via the same paths to the Tx/Rx circuits 8 of the apparatus body 2.

Then, the channel connecting section 6 of the ultrasound probe 1 switches the connections so as to move the position of the 96 channels connected to the 96 signal lines 7 by a given number to permit transmission and reception of ultrasonic waves.

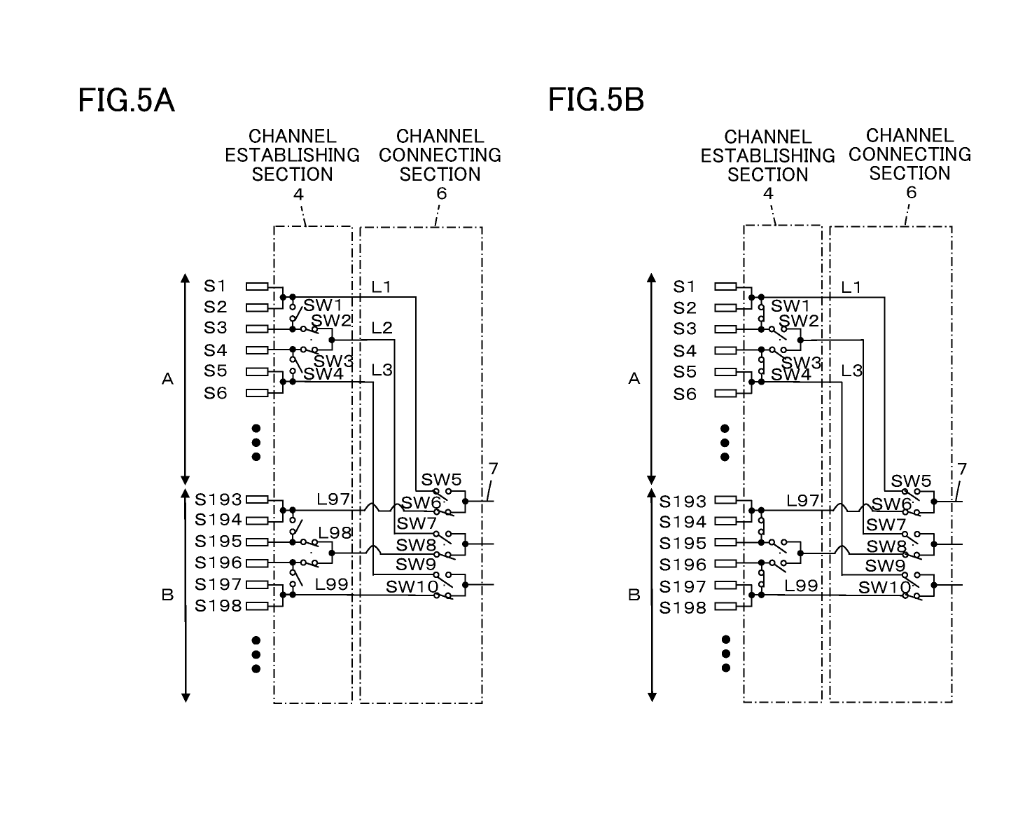

Thus, the channel connecting section 6 moves the position of the channels connected to the signal lines 7 by a given number each time the ultrasonic waves are transmitted and received until, finally, as illustrated in FIG. 5A, the switches SW5, SW7, . . . are opened while the switches SW6, SW8, . . . are closed in the channel connecting section 6 to connect 96 channels of the B group and 96 signal lines (effective signal lines). Similarly, the Tx/Rx circuits 8 of the apparatus body 2 supply the channel connecting section 6 with driving signals, which are then supplied to the B group of channels via the input/output lines L97, L98, . . . , L192 connected to the switches SW6, SW8, . . . of the channel connecting section 6. Thus, ultrasonic waves are transmitted from the B group of channels toward the subject, and the ultrasonic echoes reflected from the subject are received by the B group of channels. The reception signals thereof are inputted to the Tx/Rx circuits 8 of the apparatus body 2.

Thus, scan by the ultrasonic beams is started with the effective signal lines connected to the A group of channels, and transmission and reception of ultrasonic waves are repeated as the channels connected to the effective signal lines are moved by a given number of elements, until the effective signal lines are connected to the B group of channels to permit transmission and reception of the ultrasonic waves. The apparatus body 2 processes the reception signals inputted to the Tx/Rx circuits 8 to produce an ultrasound image and have it displayed on, for example, a monitor, not shown.

Where the ultrasound probe 1 is connected to the apparatus body 2, in which the number N of the Tx/Rx circuits 8 is 64, the switches SW1, SW4, . . . are closed and the switches SW2, SW3, . . . are opened in the channel forming section 4 as illustrated in FIG. 4B to configure the transducer array 3 composed of 128 channels each connected to three subdice elements. The 128 channels formed by the channel forming section 4 are connected to the 64 channels of the A group and the 64 signal lines (effective signal lines) connected to the Tx/Rx circuits 8 as illustrated in FIG. 4B. Then, the channel connecting section 6 switches the connections each time ultrasonic waves are transmitted and received and moves the position of the 64 channels connected to the 64 effective signal lines by a given number. Thus, as illustrated in FIG. 5B, the 64 channels of the B group are connected to the 64 effective signal lines to permit transmission and reception of ultrasonic waves, whereupon the scan by ultrasonic beams is terminated. Out of the 96 signal lines in the connection cable, the 32 signal lines (the other signals than the effective signal lines) not connected to the Tx/Rx circuits 8 of the apparatus body 2 are connected to the switches SW7, SW8, . . . (input/output lines L2, L5, . . . ) of the channel connecting section 6 that are not involved in the connections with the channels.

According to Embodiment 1, because the channel forming section 4 changes the number of channels of the ultrasound probe 1 according to the number of the Tx/Rx circuits 8 in the apparatus body 2, the ultrasound probe 1 can be adapted to a plurality of apparatus bodies 2 comprising the Tx/Rx circuits 8 of different sizes.

The number of subdice elements of each channel changed by the channel forming section 4 is not limited to 2 and 3; each channel may be composed of various numbers of subdice elements by changing the positions and the number of the switches SW for switching the connections between the subdice elements.

The channel connecting section 6 is not limited to one for performing 2:1 switching; multiplexers MUX corresponding to the time intervals at which the ultrasonic waves are transmitted and received may be used.

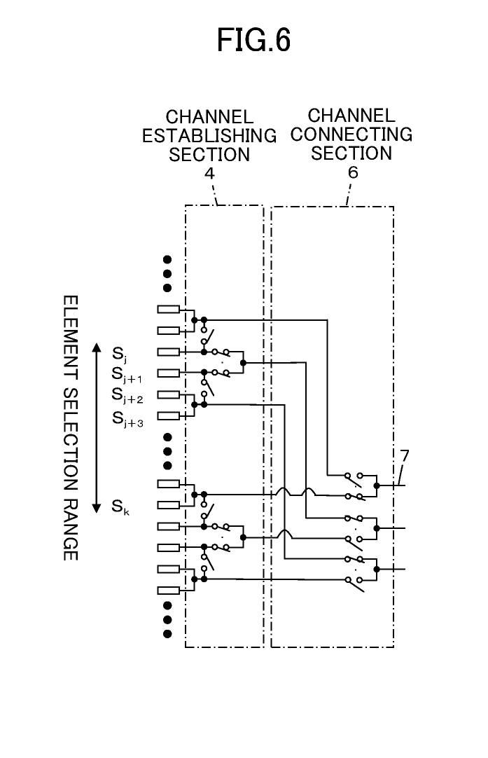

The channel connecting section 6 is not limited to one that performs switching whereby the subdice elements S1 to S384 are equally divided into two groups of the A group composed of the subdice elements S1 to S192 and the B group composed of the subdice elements S193 to S384. For example, the channel connecting section 6 may perform switching such that, as illustrated in FIG. 6, subdice elements Sⱼ to S₍ₖ₋ⱼ₊₁₉₁₎ selected from an arbitrary element selection range are connected to a plurality of signal lines 7 at the same timing. Subsequently, when the connection between the element selection range and a plurality of signal lines 7 is opened, the connections are switched to connect the subdice elements separated from each other on both sides of the element selection range to the signal lines 7. The element selection range may include any number of subdice elements; the number of subdice elements included in the element selection range and the number of those in the other ranges may be different from each other.

### Embodiment 2

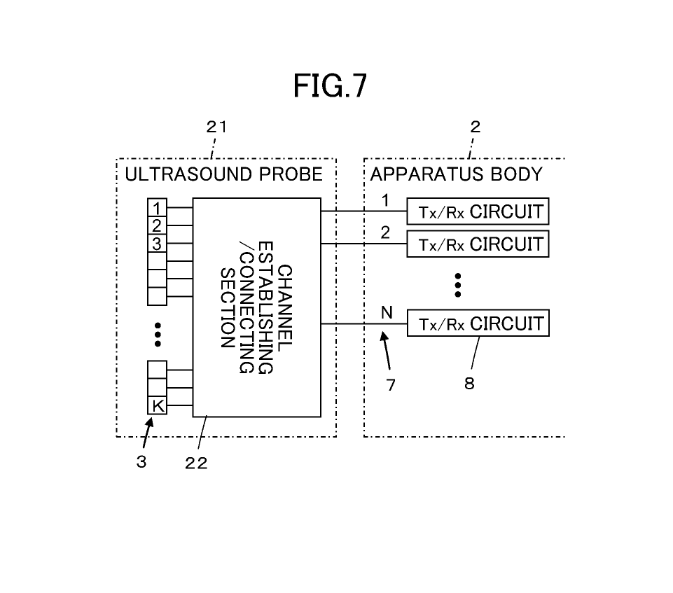

FIG. 7 illustrates a configuration of an ultrasound diagnostic apparatus according to Embodiment 2. This ultrasound diagnostic apparatus uses an ultrasound probe 21 instead of the ultrasound probe 1 of Embodiment 1 illustrated in FIG. 1. As compared with the ultrasound probe 1 according to Embodiment 1, the ultrasound probe 21 has a channel forming/connecting section 22 connected between the transducer array 3 and the signal lines 7 instead of the channel connecting section 6.

The channel forming/connecting section 22 switches the connections of a plurality of subdice elements arranged in the transducer array 3 to change the number of subdice elements constituting each of the channels and assigns a plurality of transducers constituting the transducer array 3 to a plurality of signal lines 7 connected to the Tx/Rx circuits 8 of the apparatus body 2 to form their connections.

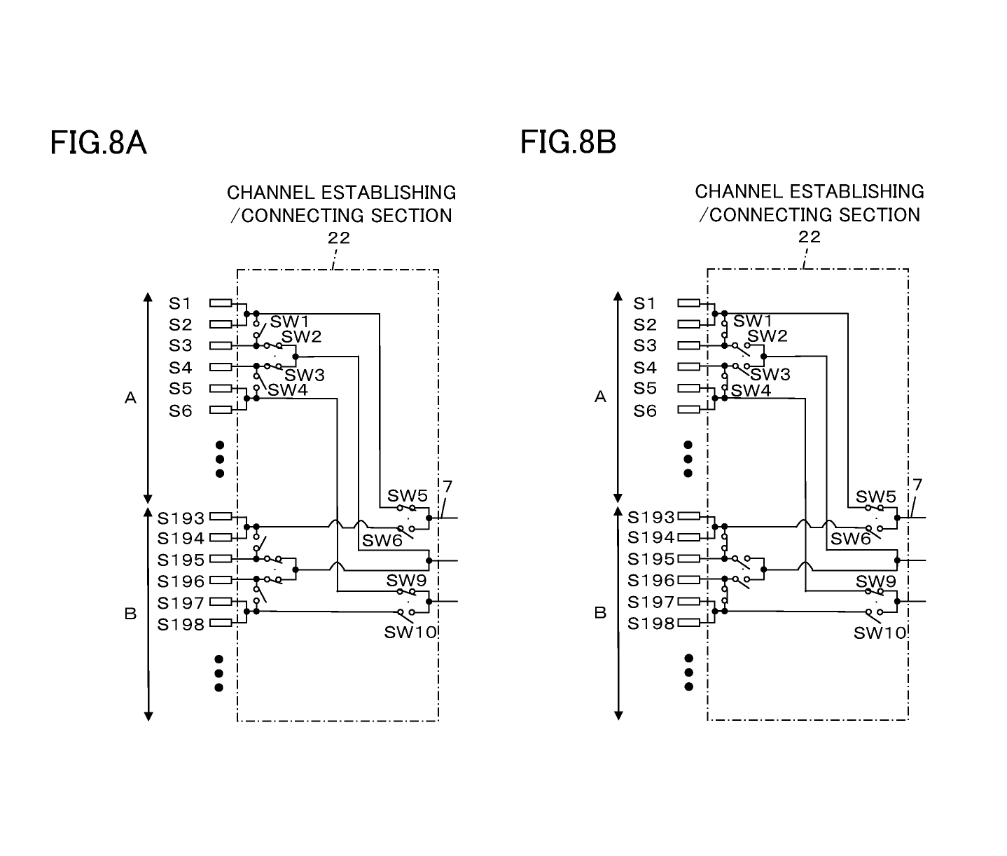

For example, where a plurality of channels are each composed of two or three subdice elements, the connections of the subdice elements constituting the channels may be switched by the channel forming/connecting section 22 as illustrated in FIGS. 8A and 8B. The channel forming/connecting section 22 comprises the switch SW1 for switching the connections between the subdice elements S1 and S2 and the subdice element S3, the switches SW2 and SW3 for switching the connection between the subdice elements S3 and S4, the switch SW4 for switching the connections between the subdice element S4 and subdice elements S5 and S6, and a plurality of other switches for likewise switching the connections between the subdice elements constituting the channels. The channel forming/connecting section 22 opens the switches SW1, SW4, . . . and closes the switches SW2, SW3, where each channel is composed of two subdice elements as illustrated in FIG. 8A and closes the switches SW1, SW4, . . . and opens the switches SW2, SW3, . . . , where each channel is composed of three subdice elements as illustrated in FIG. 8B.

The channel forming/connecting section 22 comprises the switches SW5, SW2, SW3, SW9 . . . for switching the connections between a plurality of signal lines 7 and the subdice elements S1 to S192 and the switches SW6, SW10, . . . for switching the connections between a plurality of signal lines 7 and the subdice elements 5193 to S384. Thus, out of a plurality of channels, the A group of channels composed of a number of channels obtained by equally dividing the plurality of channels are connected to the signal lines 7, and the position of the channels connected to the signal lines 7 is sequentially moved by a given number of elements as ultrasonic waves are transmitted and received. The channel forming/connecting section 22 repeats the process of moving the position of the channels connected to the signal lines 7 until the B group of channels are connected. The switches SW2, SW3, . . . switch the connections between the subdice elements constituting the respective channels and switch the connections between a plurality of signal lines 7 and the channels. Specifically, the switches SW2, SW3, . . . select connections between a plurality of subdice elements to form a plurality of channels each composed of a plurality of subdice elements and function as channel forming/connecting section for exclusively assigning two or more of a plurality of channels to the same signal line.

First, in FIG. 7, the ultrasound probe 21 where the number K of the subdice elements is 384 is connected via 96 signal lines 7 to the apparatus body 2 where the number N of the Tx/Rx circuits 8 is 96.

In the ultrasound probe 1 connected to the apparatus body 2, the switches SW1, SW4, . . . are opened and the switches SW2, SW3, . . . are closed in the channel forming/connecting section 22 as illustrated in FIG. 8A to configure the transducer array 3 composed of 192 channels each connected to two subdice elements. The switches SW5, SW2, SW3, SW9 . . . are closed and the switches SW6, SW10, . . . are opened in the channel forming/connecting section 22 to connect 96 channels constituting the A group out of the 192 channels to 96 signal lines (effective signal lines).

Upon connection of the ultrasound probe 1 and the apparatus body 2, the 96 Tx/Rx circuits 8 built in the apparatus body 2 supplies driving signals to the channel forming/connecting section 22 of the ultrasound probe 21 via the signal lines 7. The channel forming/connecting section 22 supplies the driving signals supplied from the Tx/Rx circuits 8 to the 96 channels of the A group connected to the switches SW5, SW2, . . . .

Subsequently, each time ultrasonic waves are transmitted and received, the channel forming/connecting section 22 moves the position of the 96 channels connected to the 96 signal lines 7 by a given number, until the 96 signal lines are connected to the B group of channels. The ultrasound probe 21 opens the switches SW5, SW2, SW3, . . . and closes the switches SW6, SW10, . . . in the channel forming/connecting section 22 to connect the 96 channels of the B group and the 96 signal lines. Similarly, the 96 Tx/Rx circuits 8 of the apparatus body 2 supply the channel forming/connecting section 22 with driving signals, which are then supplied to the respective channels of the B group connected to the switches SW6, SW10, .

Thus, scan by the ultrasonic beams is started with the effective signal lines connected to the A group of channels, and transmission and reception of ultrasonic waves are repeated as the channels connected to the effective signal lines are moved by a given number of elements, until the effective signal lines are connected to the B group of channels to permit transmission and reception of the ultrasonic waves.

The channel forming/connecting section 22 is not limited to one that performs switching whereby the subdice elements S1 to S384 are equally divided into two groups, the A group composed of the subdice elements S1 to S192 and the B group composed of the subdice elements S193 to S384 and may perform switching such that subdice elements Sⱼ to S₍ₖ₌ⱼ₊₁₉₁₎ selected from an arbitrary element selection range are connected to a plurality of signal lines 7 at the same timing. The element selection range may include any number of subdice elements; the number of subdice elements included in the element selection range and the number of those in the other ranges may be different from each other.

Because the channel forming/connecting section 22 thus switches the connections between the signal lines 7 connected to a plurality of Tx/Rx circuits 8 and the respective channels by group to permit transmission and reception of ultrasonic waves, the number (96) of the signal lines 7 encased in the connection cable connecting the ultrasound probe 1 and the apparatus body 2 can be reduced as compared with the number (192) of the channels, and accordingly the ease of operation of the ultrasound probe 1, for example, can be increased.

Because the switches SW2, SW3, . . . of the channel forming/connecting section 22 switch the connections between the subdice elements constituting each of the channels and switches the connections between a plurality of signal lines 7 and the respective channels, the number of switches can be reduced as compared with that required in the ultrasound probe 1 of Embodiment 1, achieving a simple configuration.

Where, on the other hand, the ultrasound probe 21 is connected to the apparatus body 2 in which the number N of the Tx/Rx circuits 8 is 64, the switches SW1, SW4, . . . are closed and the switches SW2, SW3, . . . are opened in the channel forming/connecting section 22 as illustrated in FIG. 8B to configure the transducer array 3 composed of 128 channels each connected to three subdice elements. The switches SW5, SW9, . of the channel forming/connecting section 22 assign the 128 channels to the A group or the B group each composed of 64 channels. The elements of the A group or the B group connected to the same Tx/Rx circuits 8 are connected to 64 signal lines (effective signal lines) exclusively selected by the channel forming/connecting section 22. Out of the 96 signal lines in the connection cable, the 32 signal lines (the other signals than the effective signal lines) not connected to the Tx/Rx circuits 8 of the apparatus body 2 are connected to the switches SW2, SW3, . . . that are not involved in the connections with the channels.

According to Embodiment 2, because the channel forming/connecting section 22 changes the number of channels of the ultrasound probe 21 according to the number of the Tx/Rx circuits 8 in the apparatus body 2, the ultrasound probe 21 may be adapted to a plurality of apparatus bodies 2 comprising the Tx/Rx circuits 8 of different sizes.

### Embodiment 3

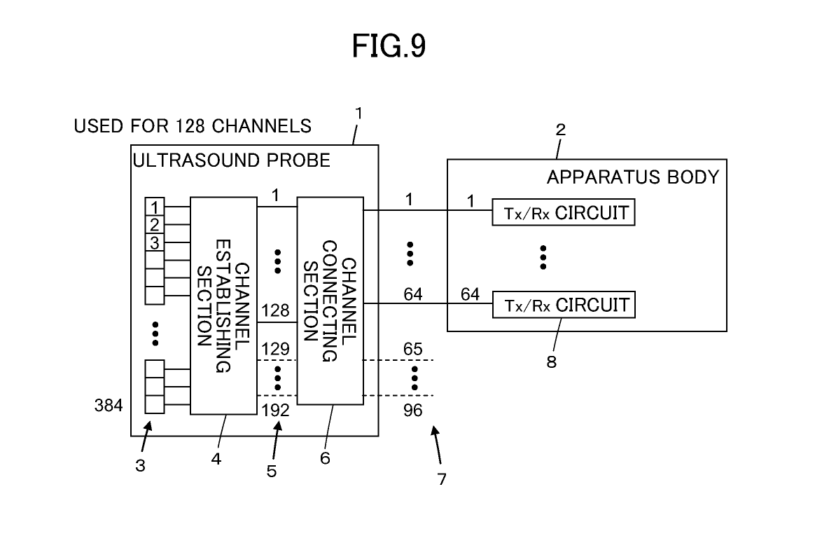

According to Embodiments 1 and 2 of the ultrasound diagnostic apparatus, one ultrasound probe may be connected to a plurality of apparatus bodies 2 for use.

Where, for example, the channel forming section 4 forms 192 channels each composed of two subdice elements in the ultrasound probe 1 comprising the 384 subdice elements and the 192 input/output lines 5 used in Embodiment 1, only 128 out of the 192 channels can be used when the ultrasound probe 1 is connected to the apparatus body 2 comprising 64 Tx/Rx circuits 8 as illustrated in FIG. 9. Specifically, out of the 192 input/output lines 5 connected to the 192 channels, 128 channels are connected to 64 Tx/Rx circuits 8 of the apparatus body via the channel connecting section 6, whereas the remaining 64 channels are not connected to the Tx/Rx circuits 8.

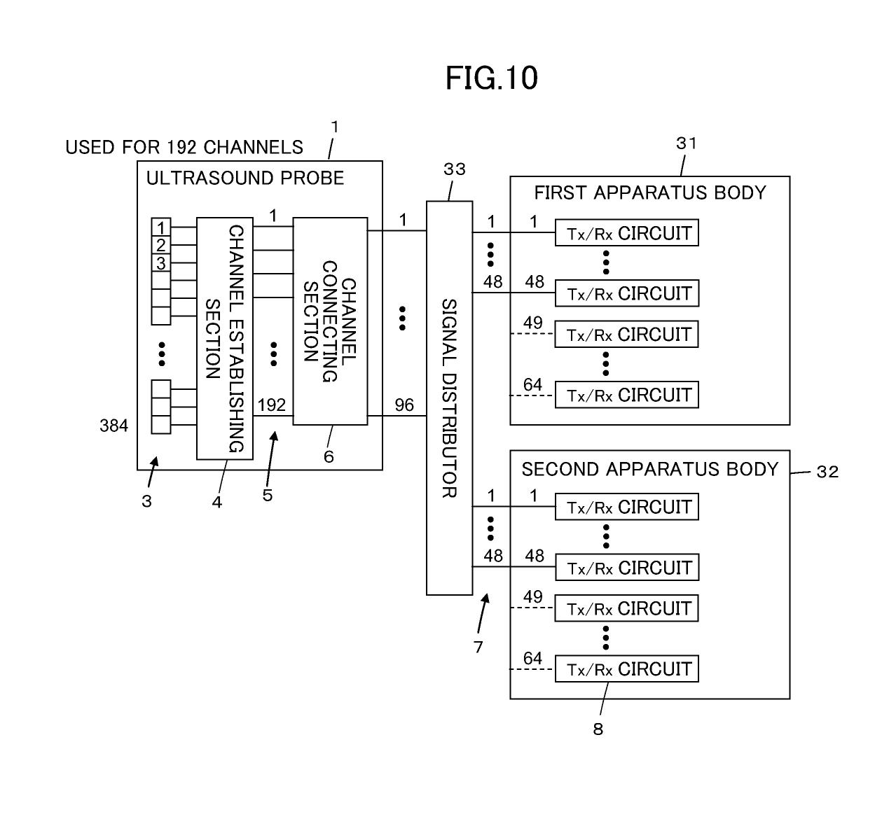

Therefore, as illustrated in FIG. 10, for example, the ultrasound probe 1 may be connected via a signal distributor 33 to a first apparatus body 31 and a second apparatus body 32 each equipped with 64 Tx/Rx circuits 8. The 192 channels each connected to two subdice elements by the channel forming section 4 are connected via 192 input/output lines 5 to the channel connecting section 6 that performs 2:1 switching. The channel connecting section 6 connects the 192 input/output lines 5 to 96 signal lines 7 and selectively connects the 96 signal lines 7 via the signal distributor 33 to 48 Tx/Rx circuits 8 each provided in the first apparatus body 31 and the second apparatus body 32. Thus, 196 channels are selectively connected to 96 Tx/Rx circuits 8 provided in the first apparatus body 31 and the second apparatus body 32. The first apparatus body 31 and the second apparatus body 32 operate in parallel at the same timing, transmitting and receiving ultrasonic waves from the 192 channels of the ultrasound probe 1.

According to Embodiment 3, even where the number of the Tx/Rx circuits of the connected apparatus body 2 is small in relation to the number of channels of the ultrasound probe 1, a high-quality ultrasound image can be obtained by operating a plurality of apparatus bodies 2 in parallel in order to increase the number of reception signals that can be simultaneously processed in parallel.

### Embodiment 4

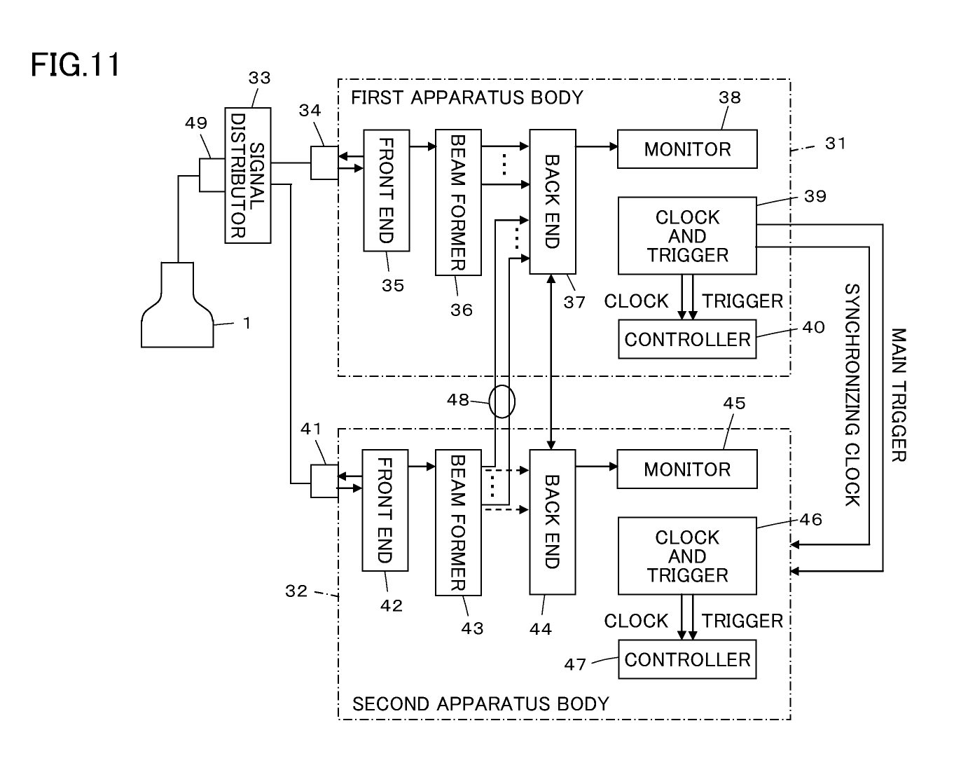

FIG. 11 illustrates internal configurations of the first apparatus body 31 and the second apparatus body 32 used in Embodiment 3 to operate two apparatus bodies in parallel. The first apparatus body 31 comprises a front end 35 connected to the signal distributor 33 via a unit side connector 34. The front end 35 is connected via a beam former 36 to a back end 37, which is connected to a monitor 38. The first apparatus body 31 further comprises a clock retrigger circuit 39, which is connected to a controller 40.

Equipped with n channels of the Tx/Rx circuits 8, the front end 35 supplies driving signals to the transducers of corresponding channels of the ultrasound probe 1, to which the front end 35 is connected via the signal distributor 33, and receives ultrasonic echoes returning from a subject to perform, for example, quadrature detection processing on reception signals generated by the transducers of these channels to produce a complex baseband signal, whereupon the front end 35 performs sampling on the complex baseband signal to produce sample data containing information on an area of a tissue. The front end 35 may produce sampling data by performing data compression processing for high efficiency encoding on the data obtained by sampling the complex baseband signal.

The beam former 36 selects one reception delay pattern from a plurality of previously stored reception delay patterns according to the reception direction set by the controller 40, and based on a selected reception delay pattern, performs the reception focusing processing by providing respective delays in the plurality of complex baseband signals represented by the sample data and adding them up. By this reception focusing processing, baseband signals (sound ray signals) in which the focal points of the ultrasonic echoes are made to converge are generated.

The back end 37 produces a B mode image signal, which is tomographic image information on the tissue of the subject, according to the sound ray signal generated by the beam former 36. The back end 37 comprises an STC (sensitivity time control) and a DSC (digital scan converter). For the sound ray signals, the STC corrects attenuation due to distance in accordance with the depth of the reflection location of the ultrasound wave. The DSC performs raster conversion of the sound ray signal corrected by the STC into an image signal compatible with the scanning method of an ordinary television signal, and then, by performing the required image processing such as contrast processing, it generates a B mode image signal.

The monitor 38 displays an ultrasound diagnostic image based on an image signal produced by the back end 37.

The clock retrigger circuit 39 supplies a clock signal to components provided in the first apparatus body 31 and supplies a trigger signal retriggered by that clock signal to components provided in the first apparatus body 31.

The controller 40 controls operations of components provided inside the first apparatus body 31.

The second apparatus body 32 also has like internal configuration as the first apparatus body 31. The second apparatus body 32 comprises a front end 42 connected to the signal distributor 33 via a unit side connector 41. The front end 42 is connected via a beam former 43 to a back end 44, which in turn is connected to a monitor 45. The second apparatus body 32 further comprises a clock retrigger circuit 46, which is connected to a controller 47.

These components provided in the second apparatus body 32 have like functions as those given the same names provided in the first apparatus body 31.

When the first apparatus body 31 and the second apparatus body 32 are in parallel operation, the first apparatus body 31, for example, is selected as master apparatus body to function as such, and the second apparatus body 32 is then selected as slave apparatus body to function as such. In this case, as illustrated in FIG. 11, the beam former 43 of the second apparatus body 32 is connected to the back end 37 of the first apparatus body 31 via the data bus 48, while the back end 44 and the clock retrigger circuit 46 of the second apparatus body 32 are connected via an operation control cable to the back end 37 and the clock retrigger circuit 39 of the first apparatus body 31.

The unit side connectors 34 and 41 connected to the signal distributor 33 are previously assigned different identification numbers (ID numbers), so that the first apparatus body 31 or the second apparatus body 32 recognizes its function as master apparatus body upon connection to the unit side connector 34 by recognizing the ID number assigned to the unit side connector 34 and recognizes its function as slave apparatus body upon connection to the unit side connector 41 by recognizing the ID number assigned to the unit side connector 41.

The probe connector 49 connected to the ultrasound probe 1 is also previously assigned an ID number that is different from those assigned to the unit side connectors 34 and 41 such that when directly connected to the probe connector 49, the first apparatus body 31 and the second apparatus body 32 recognize that they are not to perform parallel operation but independently perform normal ultrasound diagnostic operation.

Next, the parallel operation will be described. First, the signal distributor 33 ensures that the channels located in even-number positions out of a plurality of channels of the transducer array 3 of the ultrasound probe 1 are connected to the first apparatus body 31, and the channels located in odd-number positions are connected to the second apparatus body 32.

The second apparatus body 32 to function as slave apparatus body operates according to the synchronizing clock signal and the main trigger signal supplied from the clock retrigger circuit 39 of the first apparatus body 31.

When, for example, the front end 35 of the first apparatus body 31 supplies the driving signal to the transducer of the (2m+2)th channel of the ultrasound probe 1, and when the front end 42 of the second apparatus body 32 supplies the driving signal to the transducer of the (2m+3)th channel of the ultrasound probe 1, “m” being a natural number, then, upon transmission of ultrasonic waves from these two channels located adjacent to each other, the transducers of the channels of the ultrasound probe 1 having received ultrasonic echoes from the subject respectively output reception signals.

The reception signal outputted from the transducer of the channel located in an even number position in the transducer array is inputted to the front end 35 of the first apparatus body 31 to produce sample data, while the reception signal outputted from the transducer of the channel located in an odd number position in the transducer array is inputted to the front end 42 of the second apparatus body 32 to produce sample data. At this time, because the second apparatus body 32 operates according to the synchronizing clock signal and the main trigger signal supplied from the clock retrigger circuit 39 of the first apparatus body 31, the front end 35 of the first apparatus body 31 and the front end 42 of the second apparatus body 32 produce sample data at the same timing as each other.

In the first apparatus body 31, as the beam former 36 performs reception focusing processing on the sample data produced by the front end 35, a sound ray signal is produced and supplied to the back end 37. Also in the second apparatus body 32, as the beam former 43 performs reception focusing processing on the sample data produced by the front end 42, a sound ray signal is produced and supplied to the back end 37 of the first apparatus body 31 via the data bus 48.

At this time, the first apparatus body 31 and the second apparatus body 32 may be so configured as to make phase adjustment for a plurality of subdice elements constituting the individual channels of the ultrasound probe 1, combines ultrasonic beams traveling in a plurality of directions, and generates a sound ray signal based on the synthesis results.

When supplied with the sound ray signals produced respectively by the beam formers 36 and 43 of both apparatus bodies 31 and 32, the back end 37 of the first apparatus body 31 combines these sound ray signals and, based on the combined sound ray signal, produces the B-mode image signal, which is tomographic image information on the tissue of the subject. This image signal is transmitted to the monitor 38 of the first apparatus body 31, and an ultrasound diagnostic image is displayed on the monitor 38.

Because the first apparatus body 31 and the second apparatus body 32 each have an n number of channels of ultrasound transmission/reception circuits, the number of reception signals that can be simultaneously processed in parallel when these units perform a normal ultrasound diagnostic operation independently is “n”. However, when both apparatus bodies 31 and 32 perform the parallel operation, the number of reception signals that can be simultaneously processed in parallel is “2n,” which is double the number that is possible in independent operation. This enables a high quality ultrasound image to be obtained.

What is claimed is:

1. An ultrasound probe comprising:
a transducer array composed of a plurality of arrayed subdice elements;
a plurality of signal lines for connecting the transducer array to an apparatus body that controls transmission and reception of ultrasonic waves; and
a channel forming/connecting section that selects a connection between the plurality of subdice elements to form a plurality of channels each composed of a selected number of subdice elements which is changed by switching connections between a plurality of subdice elements, and assigns the plurality of channels to any of the plurality of signal lines to select, from among the plurality of signal lines, effective signal lines connected to the plurality of channels to transmit driving signals supplied to the transducer array and reception signals outputted from the transducer array to the apparatus body.

2. The ultrasound probe according to claim 1, wherein the channel forming/connecting section comprises a channel forming/connecting switches for selecting connections between the plurality of subdice elements to form the plurality of channels each composed of the selected number of subdice elements and exclusively assigning two or more of the plurality of channels to one of the signal lines.

3. The ultrasound probe according to claim 1, wherein the channel forming/connecting section comprises:
a channel forming section including a plurality of switches for selecting connections between the plurality of subdice elements to form the plurality of channels each composed of the selected number of subdice elements, and
a channel connecting section including a plurality of switches for exclusively assigning two or more channels out of the plurality of channels formed by the channel forming section to one of the signal lines,
wherein the signal lines connected by the channel connecting section to respective channels out of the plurality of signal lines are effective signal lines.

4. The ultrasound probe 1 according to claim 1, wherein the channel forming/connecting section so switches connections that the selected number of subdice elements constituting each of the channels is the same among the channels.

5. An ultrasound diagnostic apparatus comprising:
rasound probe described in claim 1 and at least one apparatus body including a plurality of transmission and reception circuits respectively connected to the effective signal lines,
wherein switching of connections between the plurality of subdice elements by the channel forming/connecting section is performed according to a total number of transmission and reception circuits of the at least one apparatus body.
